(19) United States
(12) Patent Application Publication
(10) Pub. No.: US 2005/0010271 A1
(43) Pub. Date: Jan. 13, 2005

(54) Method of using radiation to treat cutaneous and sub-cutaneous conditions

(76) Inventor: Robert F. Merchant, Reno, NV (US)

Correspondence Address: Robert C. Ryan;NATH & ASSOCIATES PLLC, 6th Floor, 1030 15th Street, N.W., Washington, DC, 20005, US

(21) Appl. No.: 10/616,122

(22) Filed: Jul. 7, 2003

(51) Int. Cl. A61N 5/06; A61B 18/20

(52) U.S. Cl. 607/89; 606/9

(57) ABSTRACT

The present application discloses a method of applying electromagnetic radiation, preferably laser radiation, most preferably at differing wavelengths, to reduce the severity of the symptoms of lipodermatosclerosis, for example. The method can reduce the severity of cutaneous and subcutaneous induration, hyperpigmentation, hyperhidrosis or other fluid weeping from the skin, erythematous, leathery appearance and texture, and associated pain and discomfort. The most preferred method involves non-invasively painting the affected area with an Aura-i laser (532 nm wavelength, generally penetrating the skin at a depth of 3-4 mm) through sapphire cooling disks and with a Lyra-i laser (1064 nm wavelength, generally penetrating the skin more deeply, such as at a depth of 6-7 mm) also through sapphire cooling disks. The method preferably involves at least three such painting applications separated by two to six, and preferably four, week intervals.

### FIELD OF THE INVENTION

This invention relates to the treatment of health disorders with electromagnetic radiation. More particularly, the present invention relates to the treatment of indurated and other cutaneous and subcutaneous conditions, such as lipodermatosclerosis, by use of electromagnetic radiation and most preferably by use of laser radiation at differing wavelengths.

### BACKGROUND OF THE INVENTION

First invented in the early 1960's, lasers are devices that use the principle of by stimulated emission and amplification of electromagnetic waves in the infrared, visible, and ultraviolet portions of the electromagnetic spectrum. The amplified electromagnetic waves in this portion of the spectrum are directed along a straight line (collimated) path and ultimately emit from this path largely in phase, resulting in a high degree of time coherence in laser radiation. Laser radiation is thus commonly referred to as “coherent,” as opposed to conventional light sources, which are generally hot bodies that radiate incoherent light by spontaneous radiation.

The focused coherency of laser radiation provides the ability to accomplish a wide variety of physical tasks and objectives with laser radiation. One common such task is the excitation of atoms and associated molecules that can absorb laser radiation at a particular frequency or wavelength.

Lasers have long been in use for treatment of various aesthetic, vein defect, and other cutaneous and subcutaneous conditions. When useable to treat these types of conditions, lasers can offer significant advantages over other health care treatments, including ease of administration of the treatment, non-invasiveness, and reduced patient discomfort.

Two prominent types of lasers that have been in this type of use for some time are the Lyra-i laser and the Aura-i laser manufactured by Laserscope of San Jose, Calif. The Lyra-i laser emits light at a wavelength of 1064 nanometers, and the Aura-i laser emits light at a wavelength of 532 nanometers.

Most commonly, the Lyra-i laser is thought to penetrate many common skin types to a depth of 6-7 millimeters. The Lyra-i laser photons (waves) are absorbed by molecules in water and de-oxygenated hemoglobin, and when the absorption rate is sufficient, the de-oxygenated hemoglobin, for example, becomes not just heated but vaporized. This phenomenon has led to use of the Lyra-i laser to non-invasively shrink the walls of subcutaneous veins and reduce or eliminate various skin and vein defect conditions. Conditions for which the Lyra-i laser has been used include superficial skin wrinkles, hair removal, red and blue leg veins, resistant red and blue facial veins, pseudo-folliculitis, and other vascular lesions of the skin and subcutaneous tissues.

The Aura-i laser is thought to penetrate many common skin types to a about half the depth of the Lyra-i laser—that is, to about 3-4 millimeters. The Aura-i laser beam photons are absorbed by molecules within water and oxygenated hemoglobin, melanin, and certain bacteria. When the absorption rate is sufficient, the absorbing hemoglobin, for example, can become heated and vaporized or at least damaged. As a result, the Aura-i laser has been used non-invasively to shrink veins and reduce or eliminate problems at shallower depths in the skin. These types of problem conditions have included wrinkle and sun damage rejuvenation, red superficial leg veins, small red facial veins, pigmented lesions in the surface of the skin and slightly below the skin (i.e., less than 1 millimeter below the skin), and vascular lesions.

Other types of electromagnetic radiation also have been used to treat some conditions of the types noted above. For example, electromagnetic radiation in the radio frequency portion of the electromagnetic spectrum has been utilized to treat varicose veins noninvasivly, by generating heat within the varicose veins and causing the veins to break down and then be replaced sufficiently by the healing process within the human body. Radio frequency radiation has also been used to treat some types of acne vulgaris.

There is a wide variety of other cutaneous and sub-cutaneous conditions and problems not known to be treatable with electromagnetic radiation, invasively or otherwise. One particularly problematic such condition is lipodermatosclerosis. Lipodermatosclerosis is a progressive fibrotic process of the skin and subcutaneous fat induced most typically by severe chronic venous insufficiency or stasis. In turn, chronic venous stasis is thought to be the result of long standing venous hypertension, leukocyte trapping with the resultant release of proteolitic enzymes, pericapillary fibrin deposits, hypofibrinolytic activity, or infections.

The lipodermatosclerosis condition is usually well defined and located in the gaiter area of the leg (at the distal medial calf just above the medial ankle bone). The condition may involve the entire circumference of the calf in this area. The affected skin can be depressed, indurated or hardened, and shiny, often with a leathery appearance and texture. The subcutaneous fat in the vicinity also typically is thickened and indurated. The hypodermis is typically indurated, adherent to the deeper layers, and in many cases may be erythematous (red and inflamed). The entire lesion is often tender to pressure and painful. Varicose veins can often be observed within this fibrotic area, and the skin above the vein may not be pigmented, although the surrounding skin is often hyperpigmented, most typically brown or erythematous.

In the acute phase of lipodermatosclerosis, patients may complain of significant pain and a hot, burning feeling in the affected area. Hyperhidrosis, or spontaneous, excessive, and possibly continuous sweating, may emanate from focal areas in the lesion. Bright red or brownish fluid (possibly lymph) may weep from the turgid skin as well. The tissues generally contract and the diameter of the ankle may narrow, strangulating the area (sclerus cuff) and accentuating venous and lymphatic stasis.

In the pertinent medical community, it has long been generally accepted that, once lipodermatosclerosis is present, the changes in the tissue are largely permanent and the associated brawny edema (indurated, hyperpigmented, and leathery areas) cannot be cured or reduced in size to any really significant degree. The object has therefore been to try to stabilize and manage the condition by trying to treat the perceived underlying problem, such as venous hypertension, varicose veins, or deep venous insufficiency. If these types of underlying problems are left untreated or the treatments are unsuccessful, the presence of lipodermatosclerosis has traditionally been a harbinger of even more serious consequences. These consequences can include ulceration, skin breakdown, spontaneous bleeding, and increased pain and discomfort.

Traditional and prevailing treatments of lipodermatosclerosis have been inconsistent. Usually, however, they involve use of drugs for treatment of hypertension or venous insufficiency, invasive surgical removal of varicose veins, or a combination of antibiotics (such as tetracycline, cehpalosporins, or erythromycim) and compression stockings or fixed external compression such as with the Circ-Aid® device. Tetracyline and pentoxyphyline also have been used to alter leucocyte function and reduce inflammatory reactions typically associated with this condition.

A recent explanation of treating lipodermatosclerosis (“LDS”) was set forth in the February, 2002, issue of Postgraduate Medicine Online:

The exact mechanism of LDS development and subsequent skin ulceration has not been defined, but it is clear that the use of graded compression stockings is essential to prevent ulcer formation and facilitate ulcer healing. An adjunctive agent that has shown remarkable success in treating LDS is stanazolol (Winstrol), an anabolic steroid that enhances fibrinolysis. The combination of stanazolol and compression stockings decreases the area of induration and pain better than compression alone. However, stanazolol is effective only for LDS, not for ulcerative disease.

Other therapies that have demonstrated minimal success include topical and systemic corticosteroids, antibiotics, cochicine, dapsone, hydroxychloroquine sulfate (Plaquenil), potassium iodide, and surgical treatment. The clinical course of LDS typically is chronic. Early recognition and treatment with compression stockings may relieve symptoms and prevent progression to ulcerative disease.

V. Iyengar, M. D., S Hsu, M. D., and J. Pielop, M. D., Progressive, painful hardening of the legs, 111 Postgraduate Medicine Online (dated February 2002).

### BRIEF SUMMARY OF THE INVENTION

The applicant has invented a method of using electromagnetic radiation to reduce symptoms such as those of lipodermatosclerosis. These symptoms may include induration, hyperhidrosis or other fluid weeping, brawny or leathery skin, hyperpigmentation, and pain, tenderness, and discomfort.

Preferably, at least some of the affected tissues are exposed to laser radiation and most preferably at multiple wavelengths. Most preferably, the radiation exposures take place repeatedly with multiple-week waiting intervals interspersed between the exposures.

Most preferably, the radiation exposure(s) is (are) accompanied or preceded by treatment of the possibly underlying condition(s) such as venous hypertension, bacterial infection, or other condition. Most preferably, the radiation exposure is non-invasive.

The applicant's most preferred radiation exposure method involves exposing the diseased tissues with one laser at one radiation wavelength and a second laser at a different radiation wavelength, then waiting two to six, and most preferably four, weeks, and then repeating this process at least one or more times.

Most preferably, one such radiation exposure penetrates the diseased tissue at least to a relatively shallow depth of 3-4 millimeters and is absorbed by at least some hemoglobin, such as oxygenated hemoglobin for example, and melanin. Preferably, the second such radiation exposure penetrates the diseased tissue at greater depths and most preferably at least 6-7 millimeters or more. Preferably, this second exposure is absorbed by at least some hemoglobin such as de-oxygenated hemoglobin for example.

It is to be understood that the foregoing is merely a brief summary of aspects of the invention. Other aspects, advantages, and objects of the invention will become apparent as the specification proceeds. The scope of the present invention is therefore to be determined by reference to the issued claims and not by whether given subject matter meets all objects or advantages set forth herein or solves, or reduces the severity of, all issues or problems in the prior art noted above.

### DETAILED DESCRIPTION OF THE PREFERRED EMBODIMENTS

As noted above, the applicant has discovered that electromagnetic radiation may successfully treat the symptoms of lipodermatosclerosis. The symptoms include induration, hyperhidrosis or other fluid weeping, brawny or leathery skin, hyperpigmentation, and pain, tenderness, and discomfort.

In the applicant's most preferred embodiment, differing wavelengths of laser radiation are swept in sequence over an area on a patient's body suffering from one or more of these symptoms. One laser wavelength penetrates the skin and underlying subcutaneous tissue to a relatively shallow depth as compared to the depth of penetration of at least a second laser wavelength applied during the treatment.

In the preferred embodiment, one laser wavelength penetrates the skin and underlying subcutaneous tissue to a depth of 3-4 millimeters and is absorbed by at least hemoglobin and melanin and most preferably also by certain bacteria such as coryne or staph bacteria for example. A second laser wavelength penetrates the same skin and underlying subcutaneous tissues to a depth substantially greater than 4 millimeters and most preferably 6-7 millimeters or more. This second laser wavelength is absorbed at least by de-oxygenated hemoglobin and possibly by certain bacteria as well.

The applicant's most preferred method utilizes the ten watt Aura-i laser although the fifteen watt Aura-i laser would work equally well of course. The Aura-i laser is a frequency doubled Nd:YAG laser, internal water-to-air cooled, wavelength 532 nm, providing 2 joules per pulse, fluence of 1-240 J/cm², pulse duration of 1-50 ms, pulse rate of up to 10 Hz, and an aim beam <5 mW @635±10 nm, adjustable.

The applicant's preferred method also utilizes the Lyra-i laser. The Lyra-i laser is a Nd:YAG laser, internal water-to-air cooled, wavelength 1064 nm, providing 40 joules per pulse, fluence of 5-900 J/cm², pulse duration of 20-100 ms, pulse rate of up to 10 Hz, and an aim beam <5 mW @635±10nm, adjustable.

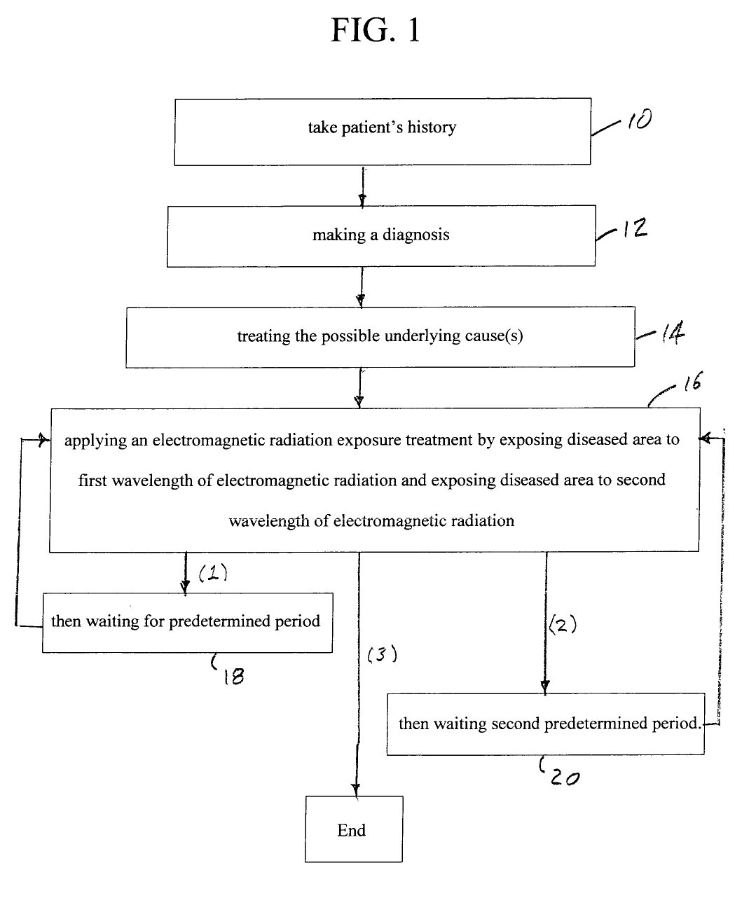

As shown in FIG. 1, when applied to treating lipodermatosclerosis, the preferred method involves the following steps.
1. taking the patient's history for a diseased area 10;
2. making a diagnosis of lipodermatosclerosis in the diseased area 12;
3. treating the possible underlying cause(s) of the lipodermatosclerosis 14;
4. applying an electromagnetic radiation exposure treatment 16 as follows:
   A. using a rapid painting application method with some overlap, using the Versatat or Dermostat Hand piece, and with direct-skin-contact sapphire disk cooling, exposing the patient's diseased area in 4 millimeter spot sizes with an Aura-i laser at an energy level ranging from 6-9 joules/cm² with a 30-40 millisecond pulse width at 1-3 pulses per second;
   B. then using a rapid painting application method with some overlap, using the Versatat or Dermostat hand piece, and with direct-skin-contact sapphire disk cooling, exposing the patient's diseased area in 10 millimeter spot sizes with a Lyra-i laser at an energy level ranging from 22-26 joules/cm² with a 30-40 millisecond pulse width at 1-3 pulses per second;
5. then waiting two to six, and preferably four weeks 18, and repeating step four above 18; and
6. again waiting two to six, and preferably four, weeks 22, and repeating step fourrebi above 20.
   Steps four through six, or portions of them, can alternatively be applied while or before performing step three.

The exact energy level to be used in step four should vary depending on skin types using the Fitzpatrick Class 1-VI classification system. For example, in step four (A), the preferred settings in jounles/cm² for these classes of skin types is as follows: I-9, II-8, III-7, IV-6, V or VI- not generally recommended. In step four (B), the preferred settings are: I-26, II and III-24-25, IV and V-22-23, VI-21-22.

The applicant has discovered that utilization of the method described above can reduce and in some cases largely or completely eliminate:
(i) induration of the diseased cutaneous and sub-cutaneous tissues;
(ii) hyperhidrosis and weeping of the diseased tissue;
(iii) brawny or leathery skin;
(iv) hyperpigmentation of the diseased area; and
(v) pain and discomfort in the diseased area.

### Example: Patient 1

Patient 1 had skin type II-III and, through steps one and two, diagnosed with liperdermatosclerosis over a substantial portion of the posterior calf of the patient's right leg. This patient's diseased area had substantial sub-cutaneous induration, erythema above the indurated tissue, hyperhidrosis, and hyperpigmentation. The patient also suffered from pain and discomfort in the diseased area,

Just prior to commencement of step four of the method above, the patient had completed step three, in this case a two year conventional course of treatment for underlying venous hypertension. The patient's lipodermatoscleroris condition was stable but included the significant problematic symptoms noted above. The preferred method steps four through six recited above were then administered to this patient with the appropriate energy levels and pulse widths in step four and with a waiting time of four weeks in steps five and six.

Three weeks after the first multiple laser wavelength treatment of step four, this patient noted improvement in sweating, discomfort, induration, and erythema. Four weeks after the second multiple laser wavelength treatment of step five, this patient's hyperhidrosis was significantly reduced. Four weeks after the third multiple laser wavelength treatment of step six, this patient's induration had softened completely in most of the affected area to resemble the softness and resiliency of the surrounding non-diseased tissues. In addition, this patient's hyperhidrosis also had cleared up completely, and the hyperpigmentation of the diseased area had improved visibly. The patient's pain and discomfort was largely eliminated.

Example: Patient 2

Patient 1 had skin type III-IV. This patient's diseased area involved the circumference of the gaitor area on the patient's right leg. This area had substantial subcutaneous induration, and erythema above the indurated tissue. The patient also suffered from pain and discomfort in the diseased area,

Just prior to commencement of step four of the method above, the patient had completed, in step three, a six month conventional course of treatment for underlying venous hypertension. The patient's lipodermatoscleroris or stasis dermatitis condition was stable but included the problematic symptoms noted above. The preferred method steps four and five were then administered to this patient with the appropriate energy levels and pulse width in step four and with a waiting time of four weeks in steps five (and with a waiting time of four weeks contemplated for step six). The exact treatment of step four, however, painted only the patient's right medial leg, sparing the lateral leg, from knee to ankle, of exposure to the laser painting of step four.

Three weeks after this patient's first multiple laser wavelength treatment in step four, the patient's entire induration, including in the lateral leg area, was significantly reduced and the overlying erythema also was nearly completely eliminated. The patient's pain and discomfort levels were also reduced throughout the diseased area. This patient has undergone a second multiple laser wavelength treatment, but this patient's results are pending at the time of preparation and filing of this application.

### Example: Patient 3

The applicant is also treating, with the preferred method described above, a skin type III-IV patient who suffers from very severe lipodermatosclerosis. After completion of step four with this third patient, the results are somewhat indefinite with possible improvement. Again, this patient's lipodermatosclerosis is quite severe—much more so than the more common severity of the first and second examples noted above.

It can thus be seen that the applicant has discovered a health care treatment technique using electromagnetic radiation, preferably laser radiation and most preferably involving exposure of multiple laser wavelengths, to greatly reduce and in some cases eliminate the symptoms of lipodermatosclerosis. In doing so, this electromagnetic radiation treatment has reduced or eliminated conditions such as induration, erythematous, hyperhidrosis or other weeping, hyperpigmentation, and pain and discomfort in the diseased area. As noted above, many of these conditions were previously thought to be largely irreversible.

This most preferred treatment is non-invasive, which can be of very significant benefit to the health care practitioner and the affected patient. The most preferred method is also believed to be very safe and easy and economical to administer. In this regard, the applicant has not observed any complications for any patient as a result of the preferred type of treatment and procedure.

The applicant believes that electromagnetic radiation treatment of lipodermatosclerosis is likely useable to treat conditions that may not qualify for a diagnosis of lipodermatosclerosis but exhibit similar symptoms. For example, electromagnetic radiation should be at least somewhat effective at reducing the severity of other types of cutaneous and subcutaneous induration, hyperhydrosis, other weeping, and associated pain and discomfort of an inflammatory or non-inflammatory nature. The radiation is most preferably laser radiation but may also involve exposure to other portions of the electromagnetic spectrum. For example, radio frequency electromagnetic spectrum may be applied as an alternative to, or in addition to, laser radiation exposure to accomplish similar objectives when desired, such as elevated heating of tissues or components of diseased tissues. The radiation is most preferably applied along with cooling of adjacent tissues or components to reduce undesired effects within them.

It should be noted, in the preferred embodiment discussed above, steps four (A) and (B) may be consolidated into one step and conducted at the same time provided that the differing wavelength laser beams are adapted to be applied at the same time and the cooling disk(s) or other cooling technique (such as liquid, liquid/air, or other cooling) can cool the exposed and adjacent tissues sufficiently to prevent undesired heat damage and unacceptable pain or discomfort for the patient.

Although the examples described in detail above involved only non-invasive application of electromagnetic, preferably laser, radiation, the procedures may be applied invasively to achieve similar or the same type of effects. For example, invasive use of the electromagnetic radiation, such as laser radiation, should be helpful in reducing the same types of symptoms in subcutaneous tissues that may otherwise be unreachable by laser radiation at the desired or required wavelength. An invasive procedure can also allow more focused exposure of tissues and reduced exposure of tissues that are not the target of the exposure.

It is to be understood that the foregoing is a detailed description of preferred embodiments. Other embodiments and variations of the preferred methods may be utilized within the scope of the present invention. The scope of the invention is therefore to be determined by reference to the accompanying claims.

In the following claims, it is to be understood that the terms “first” and “second” are used to identify differing entities but not to specify in these terms themselves that the “first” entity is necessarily “first” in time or the “second” entity is necessarily “second” in time.

1. A method of treating diseased cutaneous and sub-cutaneous tissue having symptoms of lipodermatosclerosis with laser radiation, the diseased cutaneous and subcutaneous tissue treatment method comprising the steps of:
A. exposing the diseased cutaneous and subcutaneous tissue to laser radiation absorbable by hemoglobin, with at least a substantial portion of the laser radiation penetrating said diseased cutaneous and subcutaneous tissue to a depth exceeding 4.5 millimeters below the skin surface on said diseased cutaneous tissue, said exposing of said laser radiation being for a period of time sufficient to damage hemoglobin in the diseased cutaneous and subcutaneous tissue through said depth exceeding 4.5 millimeters below said skin surface; and
B. waiting a period of time whereby healing may take place in the diseased cutaneous and subcutaneous tissue,
whereby lipodermatosclerosis symptoms in the diseased cutaneous and subcutaneous tissue may be relieved.

2. The diseased cutaneous and subcutaneous tissue treatment method of claim 1 in which said substantial portion of said laser radiation penetrate said diseased cutaneous and subcutaneous tissue to a depth exceeding 5.5 millimeters below said skin surface and wherein said exposing of said laser radiation is for a period of time sufficient to damage hemoglobin in the diseased cutaneous and subcutaneous tissue through said depth exceeding 5.5 millimeters below said skin surface

3. The diseased cutaneous and subcutaneous tissue treatment method of claim 1 also including step C: repeating steps A and B.

4. The diseased cutaneous and subcutaneous tissue treatment method of claim 3 also includes step D: again repeating at least step A.

5. The diseased cutaneous and subcutaneous tissue treatment method of claim 2 also including step C: repeating steps A and B.

6. The diseased cutaneous and subcutaneous tissue treatment method of claim 5 also includes step D: again repeating at least step A.

7. The diseased cutaneous and subcutaneous tissue treatment method of claim 1 in which step A is conducted non-invasively.

8. The diseased cutaneous and subcutaneous tissue treatment method of claim 2 in which step A is conducted non-invasively.

9. The diseased cutaneous and subcutaneous tissue treatment method of claim 4 in which step A is conducted non-invasively.

10. The diseased cutaneous and subcutaneous tissue treatment method of claim 6 in which step A is conducted non-invasively.

11. A method of treating a patient having lipodermatosclerosis in a diseased area on the patient's leg, the lipodermatosclerosis treatment method including the steps of:
A. exposing the diseased area to multiple wavelengths of laser radiation including by:
(i) exposing the diseased area with one laser having at least one laser wavelength penetrating the diseased area to a relatively shallow depth; and
(ii) exposing the diseased area with another laser having at least another laser wavelength penetrating the diseased are to a relatively deeper depth than the relatively shallow depth of the one laser; and
B. then waiting a period of time for the diseased area to heal from the exposing step A above.

12. The lipodermatosclerosis treatment method of claim 11 wherein the one laser wavelength is absorbed by oxygenated hemoglobin.

13. The lipodermatosclerosis treatment method of claim 12 wherein said another laser wavelength is absorbed by de-oxygenated hemoglobin.

14. The lipodermatosclerosis treatment method of claim 12 wherein the one laser wavelength is absorbed by oxygenated hemoglobin.

15. The lipodermatosclerosis treatment method of claim 11 wherein the one laser wavelength is absorbed by melanin.

16. The lipodermatosclerosis treatment method of claim 14 wherein said another laser wavelength is absorbed by melanin.

17. The lipodermatosclerosis treatment method of claim 11 also including step C: after completing step B repeating step A at least once.

18. The lipodermatosclerosis treatment method of claim 13 also including step C: after completing step B repeating step A at least once.

19. The lipodermatosclerosis treatment method of claim 16 also including step C: after completing step B repeating step A at least once.

20. The lipodermatosclerosis treatment method of claim 17 also including step D: after completing step C again repeating step A.

21. The lipodermatosclerosis treatment method of claim 19 also including step D: after completing step C again repeating step A.

22. A method of treating a patient having lipodermatosclerosis in a diseased area on the patient, the lipodermatosclerosis treatment method including the steps of:
A. exposing the diseased area to multiple wavelengths of laser radiation including by:
i. exposing the diseased area with a first laser having a first laser wavelength absorbed by hemoglobin and melatonin;
ii. exposing the diseased area with a second laser having a second laser wavelength absorbed by oxygenated hemoglobin; and
B. then waiting a period of time for healing to take place in the diseased area;
whereby at least lipodermatosclerosis symptoms may be reduced in the diseased area.

23. The lipodermatoscleroris treatment method of claim 22 also including as step C: after step B repeating step A at least once.

24. The lipodermatosclerosis treatment method of claim 23 wherein Step C also includes waiting a period of time for healing to take place in the diseased area and then repeating step A at least once again.

25. The lipodermatosclerosis treatment method of claim 22 wherein the first laser wavelength is 532 nm and the second laser wavelength is 1064 nm.

26. The lipodermatosclerosis treatment method of claim 23 wherein the first laser wavelength is 532 nm and the second laser wavelength is 1064 nm.

27. The lipodermatosclerosis treatment method of claim 24 wherein the first laser wavelength is 532 nm and the second laser wavelength is 1064 nm.

28. A method of treating a patient having indurated tissue, the indurated tissue treatment method including the step A of exposing the indurated tissue with laser radiation having a first laser wavelength absorbed by hemoglobin molecules, whereby the hemoglobin molecules may become heated.

29. The indurated tissue treatment method of claim 28 also including the step B of waiting for a period of time for healing of the indurated tissue and then C re-exposing the indurated sub-cutaneous tissue with laser radiation.

30. The indurated tissue treatment method of claim 28 wherein step A also includes exposing the indurated tissue with additional laser radiation having a second wavelength.

31. The indurated tissue treatment method of claim 29 wherein step A also includes exposing the indurated tissue with additional laser radiation having a second wavelength.

32. The indurated tissue treatment method of claim 29 wherein step C also includes re-exposing the indurated tissue with the additional laser radiation having the second wavelength.

33. The indurated tissue treatment method of claim 28 in which the treatment is non-invasive.

34. The indurated tissue treatment method of claim 32 in which the treatment is non-invasive.

35. A method of treating a patient having skin with excessive fluid weeping, the weeping skin treatment method including the step A of exposing the weeping skin with laser radiation having a first laser wavelength absorbed by hemoglobin molecules, whereby the hemoglobin molecules may become damaged to a degree that stimulates healing.

36. The weeping skin treatment method of claim 35 also including the step B of waiting for a period of time for healing of tissue exposed in Step A and then C re-exposing the weeping skin with laser radiation.

37. The weeping skin treatment method of claim 35 wherein step A also includes exposing weeping skin with additional laser radiation having a second wavelength.

38. The weeping skin treatment method of claim 36 wherein step A also includes exposing the weeping skin with additional laser radiation having a second wavelength.

39. The weeping skin treatment method of claim 38 wherein step C also includes re-exposing the weeping skin with additional laser radiation having the second wavelength.

40. The weeping skin treatment method of claim 35 in which the treatment method is non-invasive.

41. The weeping skin treatment method of claim 35 in which the treatment method is non-invasive.

42. The weeping skin treatment method of claim 40 in which the treatment method is non-invasive.

43. A method of treating diseased cutaneous and sub-cutaneous tissue having symptoms of lipodermatosclerosis with electromagnetic radiation, the diseased cutaneous and subcutaneous tissue treatment method comprising the steps of:
A. exposing the diseased cutaneous and subcutaneous tissue to electromagnetic radiation with at least a substantial portion of the electromagnetic radiation penetrating said diseased cutaneous and subcutaneous tissue to a depth exceeding 4.5 millimeters below the skin surface on said diseased cutaneous tissue, said exposing of said electromagnetic radiation being for a period of time sufficient to damage a component in the diseased cutaneous and subcutaneous tissue through said depth exceeding 4.5 millimeters below said skin surface; and
B. then waiting a period of time whereby healing may take place in the diseased cutaneous and subcutaneous tissue,
whereby a lipodermatosclerosis symptom in the diseased cutaneous and subcutaneous tissue may be relieved.

44. The treatment method of claim 43 wherein the exposure of step A penetrates said diseased cutaneous and subcutaneous tissue to a dept exceeding at least 5.5 millimeters below the skin surface on said diseased cutaneous tissue.

45. The treatment method of claim 43 wherein the exposure causes substantial damage to at least a portion of hemoglobin in the penetrated cutaneous and subcutaneous tissue.

46. The treatment method of claim 44 wherein the exposure causes substantial damage to at least a portion of hemoglobin in the penetrated cutaneous and subcutaneous tissue.

47. The treatment method of claim 46 wherein the exposure causes substantial damage to at least a portion of melanin in the penetrated cutaneous tissue.

48. The treatment method of claim 47 wherein exposure is applied non-invasively.
